(19) United States
(12) Patent Application Publication
(10) Pub. No.: US 2011/0034867 A1
(43) Pub. Date: Feb. 10, 2011

(54) MICROTHREAD DELIVERY SYSTEM

(76) Inventors: Jacques Guyette, Worcester, MA (US); Kevin Cornwell, Holliston, MA (US); Glenn Gaudette, Holden, MA (US); George Pins, Holden, MA (US); Marsha Rolle, Worcester, MA (US)

Correspondence Address: Duane Morris LLP - Boston;IP Department, SUITE 500, 470 ATLANTIC AVENUE, BOSTON, MA, 02210-2243, US

(21) Appl. No.: 12/532,248

(22) PCT Filed: Mar. 21, 2008
(86) PCT No.: PCT/US08/57928

§ 371 Date: Apr. 21, 2010

Related U.S. Application Data
(60) Provisional application No. 60/896,377, filed on Mar. 22, 2007.
(60) Provisional application No. 60/989,070, filed on Nov. 19, 2007.
(60) Provisional application No. 61/037,880, filed on Mar. 19, 2008.

(51) Int. Cl. A61M 37/00; A61K 35/12; A61K 9/00; A61P 9/00; C12N 11/00

(52) U.S. Cl. 604/46; 424/93.7; 424/400; 604/522; 435/174

(57) ABSTRACT

Compositions that include microthreads are provided. The compositions can be fully or partially encased in a sleeve along at least a portion of their length and can include biological cells and, optionally, therapeutic agents. Also provided are methods for using the compositions to repair or ameliorate damaged or defective tissue, including cardiovascular tissue (e.g., the myocardium).

### CROSS-REFERENCE TO RELATED APPLICATIONS

This application claims priority to U.S. Application Ser. No. 61/037,880, filed on Mar. 19, 2008; to U.S. Application Ser. No. 60/989,070, filed on Nov. 19, 2007; and to U.S. Application Ser. No. 60/896,377, filed on Mar. 22, 2007. For the purpose of any U.S. patent that may issue based on the present application, U.S. Application Ser. No. 61/037,880, U.S. Application Ser. No. 60/989,070, and U.S. Application Ser. No. 60/989,070 are hereby incorporated by reference herein in their entirety.

### TECHNICAL FIELD

This invention relates to compositions and methods useful in the repair of tissues that are ischemic or necrotic, and more particularly to compositions that include polymers that are configured as microthreads, associated with biological cells, and encased in a sleeve for delivery.

### BACKGROUND

Cardiovascular disease, which can damage both the heart and blood vessels, is the leading cause of death for both men and women in the United States and is prevalent throughout the world. Heart failure, defined as the inability of the heart to provide sufficient blood flow to body organs, affects over five million people in the U.S. alone and is the single most common diagnosis upon a patient's discharge from the hospital. Heart failure is caused by many conditions that damage the heart muscle, and there is a continuing need for therapeutic strategies that restore cardiovascular function.

### SUMMARY

The present invention is based, in part, on our discovery of various compositions that can be used to deliver cells to biological tissues. We may refer to the compositions as a whole or to one or more of their component parts as a medical device because their physical configuration and features allows them to be administered and to subsequently confer a benefit on a patient who has a damaged tissue (e.g., a tissue injured by trauma, a disease, or disorder). The underlying cause of the damage and its extent can vary, and the damage itself can be characterized as an ischemic or necrotic region, patch, or area of tissue. For example, the damaged tissue can be an ischemic area within the heart, a muscle other than the myocardium, the skin, or the brain that results from compromised blood flow and/or oxygen supply.

More specifically, the compositions can include a polymer configured as a thread or plurality of threads (which may be bundled as described below), each having a leading end and a trailing end. The threads can be encased along at least a portion of their length (e.g., along about the first or central 50%, 60%, 70%, 75%, 80%, 85%, 90%, 95%, 99%, or 100% of their length) by an open-ended sleeve. Within the sleeve, the threads may be substantially parallel with respect to one another, although a strict alignment is not required. The sleeve can be sized to accommodate various numbers of threads, whether bundled or not, and will be of sufficient strength to lend protection to the encased threads as they are drawn through a patient's tissue.

As discussed further below, and in addition to the polymer threads and surrounding sleeve (which we may refer to below as a bioreactor), the present compositions can include a plurality of biological cells and/or one or more therapeutic agents.

The leading ends of the threads, which may be bundled, can be attached to a needle or other component that facilitates movement of the threads (e.g. sleeve-covered, cell-bearing threads) into the tissue at a point within or adjacent to the damaged region of the tissue. The sleeve itself may be a gas permeable membrane made from a naturally occurring or synthetic material (e.g., an inert silicone elastomer such as a silastic gas permeable membrane).

Many different types of polymers and many combinations of polymers are useful (i.e., the threads within the sleeve may be, but are not necessarily, composed of the same types of polymers). For example, the polymer configured as a plurality of threads can include a naturally occurring polymer such as a proteoglycan, a polypeptide or glycoprotein, or a carbohydrate or polysaccharide. More specifically, the proteoglycan can be heparin sulfate, chondroitin sulfate, or keratin sulfate; the polypeptide or glycoprotein can be silk, fibrinogen, elastin, tropoelastin, fibrin, fibronectin, gelatin; and the carbohydrate or polysaccharide can be hyaluronan, a starch, alginate, pectin, cellulose, chitin, or chitosan.

One can also use synthetic polymers such as an aliphatic polyester, a poly(amino acid), poly(propylene fumarate), a copoly(ether-ester), a polyalkylene oxalate, a polyamide, a tyrosine-derived polycarbonate, a poly(iminocarbonate), a polyorthoester, a polyoxaester, a polyamidoestcr, a polyoxaester containing one or more amine groups, a poly(anhydride), a polyphosphazine, or a polyurethane. Wherein an aliphatic polyester is used, it can be a homopolymer or copolymer of: lactides; glycolides; ε-caprolactone; hydroxybuterate; hydroxyvalerate; 1,4-dioxepan-2-one; 1,5,8,12-tetraoxy-acyclotetradecane-7,14-dione; 1,5-dioxepan-2-one; 6,6-dimethyl-1,4-dioxan-2-one; 2,5-diketomorpholine; p-dioxanone (1,4-dioxan-2-one); trimethylene carbonate (1,3-dioxan-2-one); alkyl derivatives of trimethylene carbonate; δ-valerolactone; β-butyrolactone; γ-butyrolactone, ε-decalactone, pivalolactone, α,αdiethylpropiolactone, ethylene carbonate, ethylene oxalate; 3-methyl-1,4-dioxane-2,5-dione; 3,3-diethyl-1,4-dioxan-2,5-dione; or 6,8-dioxabicycloctane-7-one.

The microthreads can be “free” or can be braided, bundled, tied, or otherwise collected to form filaments. The microthreads can have a diameter of about 0.2 to 1,000 μm (e.g., about 2-100; 10-100; 20-100; 50-100; 60-100; 100-500; or 500-1,000 μm, inclusive) and, when bundled, can include about 3-300 microthreads (e.g., about 4, 10, 15, 25, or 50 microthreads).

The cells can also vary but will be cells that facilitate repair of the damaged tissue, whether through their own differentiation, integration and/or function or by promoting the survival, differentiation, integration and/or function of cells within the patient's tissues (or both). Thus, the cells associated with the microthreads can be, or can include, differentiated cells such as myocytes, epithelial cells, endothelial cells, fibroblasts, and neurons. The cells can also be stem cells, precursor cells, or progenitor cells (i.e., any cells that are not fully or terminally differentiated). Further, the stem cell can be an adult stem cell (e.g., a mesenchymal stem cell (e.g., a human mesenchymal stem cell or hMSC), an endothelial stem cell, a hematopoietic stem cell, or an adult stem cell from any other source or lineage) or an embryonic stem cell. The source of the cells can also vary. For example, the cells may be, or may include, those obtained from the same patient who is subsequently treated with the composition (i.e., the cells can be autogeneic) or they may be obtained from another person (i.e., the cells can be allogeneic).

Where a therapeutic agent is included, it may be any type of agent of facilitates repair of the patient's tissue, either directly or indirectly, or confers some other benefit on the patient. For example, the therapeutic agent can be a protein-based agent such as a polypeptide growth factor or an antibody; a vitamin or a mineral; an antimicrobial agent (e.g., an anti-viral, anti-fungal, or antibiotic), or a small organic molecule. The therapeutic agent can affect the cells within the present compositions and/or the cells within the patient's own tissues. Suitable growth factors include VEGF, an IGF (e.g., IGF-1), a PDGF, an EGF, an NGF, a BDNF, or a metalloprotease.

In addition to the compositions per se, the present invention features methods of making cell-containing compositions that can be used to deliver cells to a patient. To make those compositions, one can place the microthreads described herein in a cell culture vessel with cells such that the cells become associated with the plurality of threads to form the cell-containing compositions. The precise nature of the association can vary. The cells can associate with the microthreads just as they would with any other biocompatible or inert substrate. In culture, the sleeve may be placed around the microthreads before or after the cells are added. We may refer to the sleeve in the Examples below as a sheath or bioreactor.

Methods of treatment are also features of the present invention. For example, one can treat a patient who has ischemic or necrotic tissue by administering a composition described herein to the ischemic or necrotic tissue. More specifically, one can pierce the tissue (e.g., with a needle or other tapered object that may be attached at an end of the microthreads) and draw a cell-containing composition into the tissue. The sleeve and needle can then be removed. In some embodiments, the sleeve can also be tapered. For example, the sleeve can be tapered so that that it narrows toward the leading end of the microthreads contained therein. Thus, the sleeve or a terminal portion of the sleeve can be conical, with the apex or narrower portion of the cone approaching the needle or tissue-piercing element by which the sleeve-enclosed microthreads are drawn into the tissue. The ischemic or necrotic tissue can be myocardial tissue.

In one embodiment, the invention features methods of delivering a biological cell to a tissue in need of repair (whether due to tissue loss or malfunction due, for example, to an inadequate supply of blood and/or oxygen). The tissue can be cardiac tissue (including the myocardium per se), other muscle (e.g., skeletal muscle), skin, or a soft tissue such as the tissue of an internal organ such as the pancreas, kidney, spleen, liver, or lung. The steps of the method can include:

(a) providing a biopolymer thread (or a plurality thereof) comprising or associated with one or more biological cells, wherein the thread is attached to a surgical needle and at least a portion of the thread is encased within a sleeve;

(b) drawing the needle through a region of the tissue to insert the sheath within the tissue: and

(c) removing the needle and sheath, thereby retaining the biopolymer thread in the tissue.

The needle can be a conventional needle (e.g., a pointed stainless steel needle such as those usually contained in a suture pack), which may vary in size and may be straight or curved. The microthreads can be directly or indirectly attached to the needle (e.g., a linker such as silk or nylon may be used to attach the microthreads to the base of the needle), and the sleeve can be attached to the needle or the linker to help ensure that the sleeve is drawn smoothly through the tissue.

The invention further encompasses methods of making a tissue repair composition comprising the microthreads described herein, means for drawing the microthreads through a tissue (e.g., a needle), and means for reducing the stress that would otherwise be applied to the microthreads by the tissue (e.g., a sleeve). These methods can include the steps of:

(a) providing or introducing cells that induce or enhance the repair or regeneration of tissue into a culture medium comprising a polymer thread (or a plurality of threads configured as a bundle) having a leading end and a trailing end;

(b) culturing the cells under conditions that allow the cells to associate with the thread; and

(c) removing the thread and associated cells from the culture medium.

Alternatively or in addition to the cells, the microthreads can be used to deliver a therapeutic agent, examples of which are provided further below.

In the production and treatment methods, the polymer thread can be encased within a sleeve, and the leading end of the polymer thread can be attached to a needle. The sleeve can be complete around its circumference (e.g., as an intact tube), perforated, or partially open along the longitudinal axis. For example, the sleeve can include a conical leading edge and body that is semi-circular and therefore partially encases the enclosed microthreads.

Where the polymer is, or includes, a fibrin microthread, the fibrin microthread can be made by a method that includes the steps of:

(a) providing fibrinogen and a sufficient amount of a molecule capable of forming fibrin from the fibrinogen (the fibrin-forming molecule can be a serine proteases (e.g., thrombin, which may be in a mutant form that exhibits increased or decreased enzymatic activity); and

(b) extruding a mixture of the fibrinogen and the molecule through an orifice into a medium thereby producing a fibrin microthread.

The fibrinogen can be human fibrinogen or fibrinogen of a non-human primate, a domesticated animal, or a rodent. The fibrinogen can also be obtained from a naturally occurring source or can be recombinantly produced. The molecule present during extrusion can be thrombin.

Cells can be included in the process so that they are extruded together with the fibrinogen. Additional cells or other therapeutic agents may be added in addition to the cells incorporated by joint extrusion.

While the invention is not so limited, it is our expectation that this microthread-based delivery system will provide targeted delivery, resulting in concise placement of cells in a region of interest. We anticipate that the protective sleeve will increase cell attachment to the microthreads, improving viability during the delivery phase and enhancing cell engraftment in the tissue. Our compositions further allow the ability to concurrently deliver therapeutic proteins and growth factors incorporated into the microthreads to enhance tissue regeneration. As such, these cell-seeded microthreads serve as a platform technology for efficiently delivering viable cells to tissues such as the infarcted myocardium and for precisely directing cellular function. Furthermore, the microthreads may promote myocyte alignment during myocardial regeneration.

Other features of the present inventions will be described below and are illustrated in the accompanying drawings.

### DESCRIPTION OF DRAWINGS

FIG. 1 is a schematic diagram illustrating an hMSC seeded microthread and delivery device.

FIG. 2 is a panel of photomicrographs illustrating tracking stem cells with quantum dots (QDs).

FIG. 3 is a schematic diagram illustrating a fibrin extrusion device.

FIG. 4 is a panel of scanning electron micrographs of biopolymer microthreads. (A) self-assembled collagen microthreads, (B) fibrin biopolymer microthreads and (C) a cell-seeded fibrin thread.

FIG. 5 is a schematic of attachment-assay incubation chamber.

FIG. 6 is a panel of photomicrographs illustrating hMSCs on fibrin microthreads over time.

FIG. 7 is a panel of photographs illustrating fibrin microthread delivery to canine myocardium.

FIG. 8 is a schematic diagram concerning cell seeding capacity.

FIG. 9 illustrates the HDM method of the invention.

### DETAILED DESCRIPTION

Recent evidence suggests that the delivery of human mesenchymal stem cells (hMSCs) to the infarcted heart improves mechanical function in both clinical and experimental animal studies, although the functional mechanism remains equivocal. A major limitation of cell delivery systems for cardiac repair has been ineffective localization, and persistence and retention of a physiologically relevant number of cells in the heart. Recently, we developed new methods for producing biopolymer microthreads that can be tailored to modulate cell attachment and migration. Further, we have demonstrated that we can precisely track the location of cells delivered to myocardium using a novel quantum dot based tracking method. Based on these observations, we describe herein cell-seeded (e.g., hMSC-seeded) microthreads that enhance targeted cell delivery to tissues including infarcted regions of the heart.

Cardiac myocytes have long been thought to be terminally differentiated and lacking in the ability to proliferate. However, recent data suggested that myocytes may re-enter the cell cycle in regions bordering a myocardial infarction (Beltrami et al., N. Engl. J. Med. 344:1750-1757, 2001). These data demonstrated that approximately 4% of the myocytes in the borderzone (between infarcted and viable tissue) were positive for Ki-67, a nuclear molecule involved in cell proliferation. Since this report, other investigators also documented myocyte proliferation in various environments. Schuster and colleagues induced endogenous myocyte proliferation in a rat infarct model by delivering human endothelial progenitor cells (Schuster et al., Am. J. Physiol. Heart Circ. Physiol. 287:H525-32, 2004). Using a rat specific antibody to Ki-67 they assured that native rat myocytes entered the cell cycle and not the human cells that were delivered to the myocardium. Recently, p38 MAP kinase inhibition has also been shown to allow adult cardiomyocytes to proliferate in vitro (Engel et al., Genes Dev., 2005). Accordingly, agents that inhibit p38 MAP kinase can be incorporated in the present compositions and methods (e.g., delivered to the myocardium via the present microthreads).

Stein cells releasing paracrine factors may also induce native myocytes to proliferate and such factors can also be incorporated (see Doronin et al., Keystone Symposium: Molecular Biology of Cardiac Diseases and Regeneration, 2005). The release of paracrine factors from endogenous or delivered stem cells may simulate the signaling environment of the fetal mammalian heart and may enhance the ability of native myocytes to divide (Chien et al., Science 306:239-240, 2004). This mechanism may be responsible for the regeneration associated with delivery of mesenchymal stein cells to the infarcted heart (Mazhari and Hare, Nat. Clin. Pract. Cardiovasc. Med. 4 Suppl. 1:S21-6, 2007).

There is still debate as to whether progenitor cells differentiate into functional cardiac myocytes (see Beltrami et al., Cell 114:763-776, 2003; Oh et al., Ann. N.Y. Acad. Sci. 1015:182-189, 2004; Laugwitz et al., Nature 433:647-653, 2005; Murry et al., Nature 428:664-668, 2004; and Balsam et al., Nature 428:668-673, 2004).

Initial clinical trials with stem cells delivered into damaged myocardium yielded some positive results. Strauer and associates demonstrated the clinical feasibility of using bone marrow derived cells to treat myocardial infarction (Circulation 106:1913-1918, 2002). These investigators reported a decrease in the infarct developed after acute myocardial infarction in patients who received cell therapy. However, safety issues, particularly with respect to in-stent restenosis, have been raised (Kang et al., Lancet 363:751-756, 2004). As it seems that many different types of cells (including non-stem cells) can improve cardiac function (Murry et al., J. Am. Coll. Cardiol. 47:1777-1785, 2006; see also Gaudette and Cohen, Circulation 114:2575-2577, 2006), our compositions and methods can be practiced with a variety of cell types, as described further herein.

Current methods for delivering progenitor cells to the heart include intravascular (IV), intracoronary (IC) and intramyocardial (IM). While IV delivery of cells is the least invasive, most of the cells get trapped in the lungs (Kraitchman et al., Circulation 112:1451-1461, 2005), with less than 1% of the cells residing in the infarcted heart (Barbash et al., Circulation 108:8630868, 2003). During angioplasty, cells can be delivered IC directly to the region of interest. However, upon restoration of blood flow the majority of cells are washed away from the region of interest and only 3% of the delivered cells are engrafted into the heart (Hou et al., Circulation 112:1150-1156, 2005). The IM route for injection of cells resulted in 11% of the cells engrafting in the heart (Hou et al., supra).

While many researchers have developed tissue constructs that incorporate fetal or neonatal rat cardiac myocytes into engineered cardiac tissue (see Radisic et al., Philos. Trans. R. Soc. Lond. B. Biol. Sci. 362:1357-1368, 2007), a limited number of investigators have researched scaffold-based strategies for delivering stem cells to the heart, including alginate (Leor et al, Heart 93:1278-1284, 2007), collagen (Simpson et al. Stem Cells 25:2350-2357, 2007), collagen/GAG (Xiang et al, Tissue Eng. 112:2467-2478, 2006), and Matrigel (Zimmermann et al, Biomaterials 25:1639-1647, 2004; Laflamme et al, Nat. Biotechnol. 25:1015-1024, 2007). However, stem cells delivered via scaffolds appear to have a difficult time transversing the myocardial wall to reach the endocardium (Simpson et al, supra), where most clinical myocardial infarctions reside. Recently, Simpson and colleagues, using a scaffold-based delivery vehicle, demonstrated that only 1% of engrafted hMSCs were found in the endocardial space. Thus, using current methods, it is difficult to efficiently deliver a large number of stem cells to a well defined region.

Biopolymer threads are a class of fibrous scaffolding materials manufactured from repeating subunits of naturally derived molecules including proteins such as silk, collagen, chitosan and alginate. These fibrous materials are biodegradable and exhibit a broad range of mechanical and biochemical properties that can be tuned to meet specific applications including the regeneration of cartilage, tendon, ligament, and skin. Additionally, thread-based scaffold morphology directs the alignment of cells and cytoskeletal components, ultimately leading to aligned matrix deposition and tissue regeneration (Rovensky et al, J. Cell Sci. 107:1255-1263, 1994; Canty et al., J. Biol. Chem. 281:38592-38598, 2006; and Silver et al., J. Biomech. 36:1529-1553, 2003).

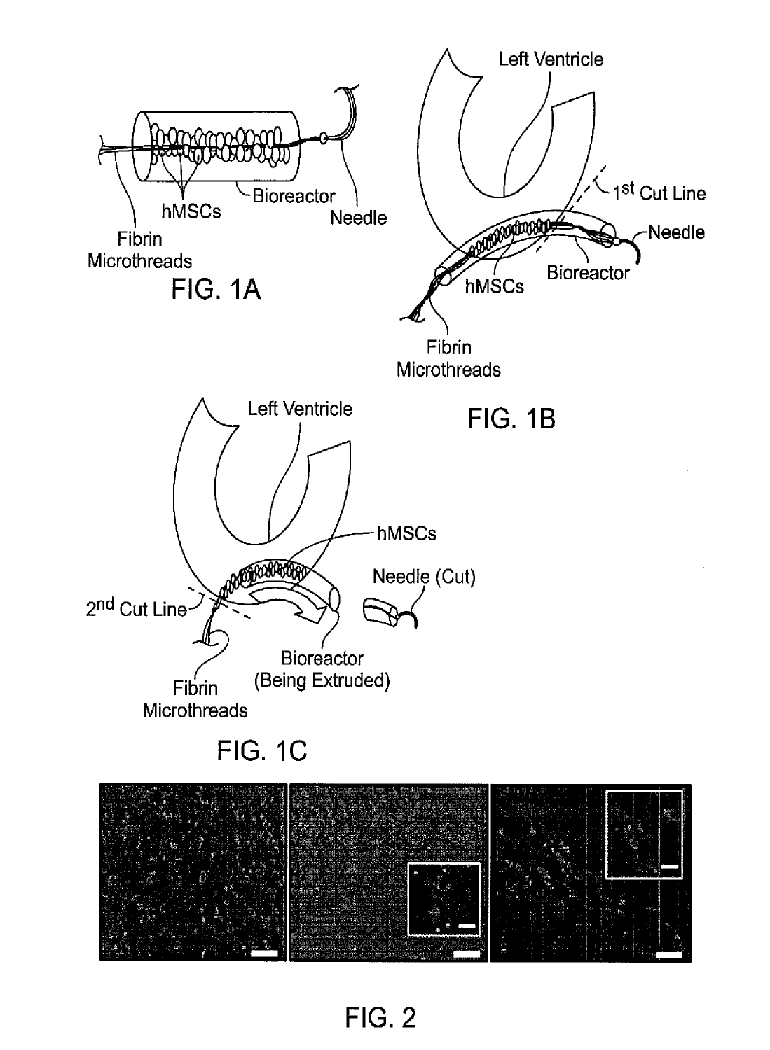

Delivery of the present microthreads can be facilitated by whole or partial enclosure within a sleeve or bioreactor, which may be gas permeable and can serve as a protective shield during deployment to a tissue (FIG. 1).

To help optimize the conditions for producing the present compositions, we have incubated quantum dot loaded hMSCs on fibrin microthreads encapsulated within a gas permeable bioreactor for various periods of time. We can assess hMSC “stemness,” as an indication of the pluripotency of the cells, morphology, cell density, and viability. Concurrently, we studied the mechanical strength of the cell-seeded microthreads and deployed into the beating rat heart. Cell engraftment can be assessed in such an animal model at various times (e.g., 0 and 3 days) post implantation.

The ability of the present compositions to improve tissue function can also be studied in animal models by assessing engraftment and resultant regional function in an infarcted heart. For example, myocardial infarction can be induced by temporary ligation of the left anterior descending artery in athymic rats. Cell-seeded microthreads can be delivered to the infarcted myocardium to span the region of the infarct and the peri-infarct border, and animals can be sacrificed after various periods of time (e.g., 1, 7 and 28 days) for assessment of regional mechanical function and histological evaluation of hMSC localization, viability, proliferation, engraftment, and differentiation within the infarcted heart.

Regarding the myocardial injury model, one can use athymic male Sprague-Dawley rats (rh mu-mu, Harlan). The rats can be anesthetized with ketamine/xylazine intraperitoneally, intubated, maintained on isofluorane inhalation (1.5-2%) and mechanically ventilated with room air supplemented with oxygen. A left thoracotomy can then be performed to expose the left ventricle, and the left anterior descending artery (LAD) can be visualized using a dissecting microscope. A 7-0 prolene suture (Ethicon, Johnson and Johnson) will be passed through the ventricular wall to create a temporary ligature around the LAD to induce myocardial ischemia. After one hour of ischemia, the ligature is released to restore perfusion to the left ventricle. Following reperfusion, a composition as described herein (e.g., an hMSC-loaded and sheathed bundle of microthreads) can be delivered to the infarct area. hMSCs suspended in serum-free medium, microthreads without cells, or serum-free medium alone can be used as controls. Immediately after hMSC delivery, the chest is be sutured closed layer-by-layer and the animals are placed in a heated chamber and allowed to recover under supervision.

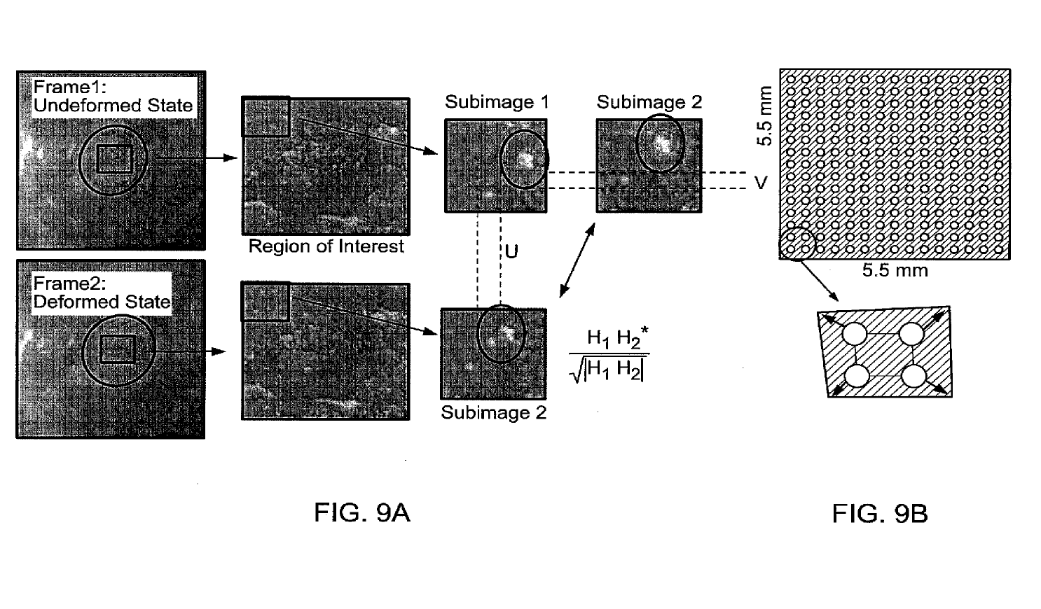

Provided herein are methods of making polymer-based compositions (e.g., fibrin microthreads), populating those compositions with biological cells and/or therapeutic agents, and using those compositions to repair tissue. Also encompassed are methods of high density mapping as described further below and illustrated in FIG. 9. While the repair can be carried out in vivo, the present compositions can also be used to treat tissue generated or maintained in cell culture or tissue that has been harvested for transplantation.

The production methods can include providing fibrinogen and a sufficient amount of a molecule capable of forming fibrin from the fibrinogen; and extruding a mixture of the fibrinogen and the molecule through an orifice into a medium thereby producing a fibrin microthread. The molecule is a protease, for example, thrombin. The medium can be a buffered solution having a pH of about 6.0 to about 8.0; a suitable pH is about 7.4. The fibrin microthreads are formed by coextruding a solution of fibrinogen, the fibrin precursor, with one or more molecules capable of forming fibrin, under conditions suitable for fibrin formation, into an aqueous buffered medium, incubating the extruded solution until filament formation is observed, and then drying the filaments. During the extrusion process, the fibrinogen is cleaved to generate fibrin monomers which self-assemble in situ to form filaments.

Polypeptides: The terms “polypeptide” and “peptide” are used herein to refer to a compound of two or more subunit amino acids, amino acid analogs, or other peptidomimetics, regardless of post-translational modification (e.g., amidation, phosphorylation or glycosylation). The subunits can be linked by peptide bonds or other bonds such as, for example, ester or ether bonds. The term “amino acid” refers to natural and/or unnatural or synthetic amino acids, which may, as noted above, be D- or L-form optical isomers. Full-length proteins, analogs, mutants, and fragments thereof are encompassed by this definition.

Fibrinogen: The fibrin component of the fibrin microthreads is a proteolytic cleavage product of fibrinogen. Fibrinogen, a soluble protein typically present in human blood plasma at concentrations between about 2.5 and 3.0 g/L, is intimately involved in a number of physiological processes including hemostasis, angiogenesis, inflammation and wound healing. Fibrinogen is 340,000 Da hexameric glycoprotein composed of pairs of three different subunit polypeptides, Aα, Bβ, and γ, linked together by a total of 29 disulfide bonds. During the normal course of blood coagulation, the enzyme thrombin cleaves small peptides from the Aα and Bβ chains of fibrinogen to generate the insoluble fibrin monomer. The fibrin monomers self-assemble in a staggered overlapping fashion through non-covalent, electrostatic interactions to form protofibrils; the protofibrils further assemble laterally into thicker fibers that ultimately intertwine to produce a clot. Fibrinogen is expressed primarily in the liver, although low levels of extrahepatic synthesis have been reported for other tissues, including bone marrow, brain, lung and intestines. The thrombin catalyzed conversion of fibrinogen to fibrin is common to all extant vertebrates; accordingly, the amino acid sequence of fibrinogen is highly conserved evolutionarily. Each polypeptide subunit is the product of a separate but closely linked gene; multiple isoforms and sequence variants have been identified for the subunits. Aminoa acid sequences for the fibrinogen subunits are in the public domain. The fibrinogen Aα polypeptide is also known as fibrinogen α chain polypeptide; fibrinogen a chain precursor; Fib2; MGC119422; MGC119423; and MGC119425. The fibrinogen Bβ polypeptide is also known as fibrinogen β chain polypeptide; fibrinogen β chain preproprotein.; MGC104327; and MGC120405 and the fibrinogen γ polypeptide is also known as fibrinogen γ chain polypeptide and fibrinogen γ chain precursor.

Any form of fibrinogen that retains the ability to function (e.g., retains sufficient activity to be used for one or more of the purposes described herein) may be used in the manufacture of the fibrin microthreads. The fibrinogen is human fibrinogen or fibrinogen of a non-human primate, a domesticated animal, or a rodent. The fibrinogen is obtained from a naturally occurring source or is recombinantly produced. All that is required is that the fibrinogen retains the ability to form polymerized fibrin monomers and that the fibrin microthreads prepared from those fibrin monomers retain, or substantially retain, the capacity to support cell attachment and proliferation. The amino acid sequence of fibrinogen subunit polypeptides can be identical to a standard reference sequence in the public domain. As noted, the present invention includes biologically active variants of fibrinogen subunit polypeptides, and these variants can have or can include, for example, an amino acid sequence that differs from a reference fragment of a fibrinogen subunit polypeptide by virtue of containing one or more mutations (e.g., an addition, deletion, or substitution mutation or a combination of such mutations). One or more of the substitution mutations can be a substitution (e.g., a conservative amino acid substitution), with the proviso that at least or about 50% of the amino acid residues of the variant are identical to residues in the corresponding wildtype fragment of a fibrinogen subunit polypeptides. For example, a biologically active variant of a fibrinogen subunit polypeptides can have an amino acid sequence with at least or about 50% sequence identity (e.g., at least or about 50%, 55%, 60%, 65%, 70%, 75%, 80%, 85%, 90%, 95%, 97%, 98%, or 99% sequence identity) to a fibrinogen subunit polypeptide. Conservative amino acid substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine. Alternatively, any of the components can contain mutations such as deletions, additions, or substitutions. All that is required is that the variant fibrinogen subunit polypeptide have at least 5% (e.g., 10%, 20%, 30%, 40%, 50%, 60%, 70%, 80%, 90%, 95%, 99%, 100%, or even more) of the ability of the variant fibrinogen subunit polypeptide containing only the reference sequences to retains the ability to form polymerized fibrin monomers and that the fibrin microthreads prepared from those fibrin monomers retain, or substantially retain, the capacity to support cell attachment and proliferation.

The fibrinogen may be obtained from any of a wide range of species. It is not necessary that the fibrinogen be from a species that is identical to the host, but should simply be amenable to being remodeled by invading or infiltrating cells such as differentiated cells of the relevant host tissue, stem cells such as mesenchymal stem cells, or progenitor cells. The fibrinogen useful for the invention can optionally be made from a recipient's own tissue. Furthermore, while the fibrinogen will generally have been made from one or more individuals of the same species as the recipient of the fibrin microthreads, this is not necessarily the case. Thus, for example, the fibrinogen can be derived from bovine tissue and be used to make fibrin microthreads that can be implanted in a human patient. Species that can serve as recipients of fibrin microthreads and fibrinogen donors for the production of fibrin microthreads can include, without limitation, mammals, such as humans, non-human primates (e.g., monkeys, baboons, or chimpanzees), pigs, cows, horses, goats, sheep, dogs, cats, rabbits, guinea pigs, gerbils, hamsters, rats, or mice.

The fibrinogen may be partially or substantially pure. The term “substantially pure” with respect to fibrinogen refers to fibrinogen that has been separated from cellular components by which it is naturally accompanied, such that it is at least 60% (e.g., 70%, 80%, 90%, 95%, or 99%), by weight, free from polypeptides and naturally-occurring organic molecules with which it is naturally associated. In general, a substantially pure polypeptide will yield a single major band on a non-reducing polyacrylamide gel. A substantially pure polypeptide provided herein can be obtained by, for example, extraction from a natural source (e.g., blood or blood plasma from human or animal sources, e.g., non-human primates (e.g., monkeys, baboons, or chimpanzees), pigs, cows, horses, goats, sheep, dogs, cats, rabbits, guinea pigs, gerbils, hamsters, rats, or mice), chemical synthesis, or by recombinant production in a host cell.

The fibrinogen can include post-translational modifications, i.e., chemical modification of the polypeptide after its synthesis. Chemical modifications can be naturally occurring modifications made in vivo following translation of the mRNA encoding the fibrinogen polypeptide subunits or synthetic modifications made in vitro. A polypeptide can include one or more post-translational modifications, in any combination of naturally occurring, i.e., in vivo, and synthetic modifications made in vitro. Examples of post-translational modifications glycosylation, e.g., addition of a glycosyl group to either asparagine, hydroxylysine, serine or threonine residues to generate a glycoprotein or glycopeptides. Glycosylation is typically classified based on the amino acid through which the saccharide linkage occurs and can include: N-linked glycosylation to the amide nitrogen of asparagines side chains, O-linked glycosylation to the hydroxyloxygen of serine and threonine side chains, and C-mannosylation. Other examples of pot-translation modification include, but are not limited to, acetylation, e.g., the addition of an acetyl group, typically at the N-terminus of a polypeptide; alkylation, e.g., the addition of an alkyl group; isoprenylation, e.g., the addition of an isoprenoid group; lipoylation, e.g. attachment of a lipoate moeity; phosphorylation, e.g., addition of a phosphate group to serine, tyrosine, threonine or histidine; and biotinylation, e.g., acylation of lysine or other reactive amino acid residues with a biotin molecule.

Fibrinogen can be purified using any standard method know to those of skill in the art including, without limitation, methods based on fibrinogen's low solubility in various solvents, its isoelectric point, fractionation, centrifugation, and chromatography, e.g., gel filtration, ion exchange chromatography, reverse-phase HPLC and immunoaffinity purification. Partially or substantially purified fibrinogen can also be obtained from commercial sources, including for example Sigma, St. Louis Mo.; Hematologic Technologies, Inc. Essex Junction, VT; Aniara Corp. Mason, Ohio.

Fibrinogen can also be produced by recombinant DNA techniques. Nucleic acid segments encoding the fibrinogen polypeptide subunits can be operably linked in a vector that includes the requisite regulatory elements, e.g., promoter sequences, transcription initiation sequences, and enhancer sequences, for expression in prokaryotic or eukaryotic cells. Methods well known to those skilled in the art can be used to construct expression vectors containing relevant coding sequences and appropriate transcriptional/translational control signals. Alternatively, suitable vector systems can be purchased from commercial sources.

Nucleic acid segments encoding the fibrinogen polypeptide subunits are readily available in the public domain. The terms “nucleic acid” and “polynucleotide” are used interchangeably herein, and refer to both RNA and DNA, including cDNA, genomic DNA, synthetic DNA, and DNA (or RNA) containing nucleic acid analogs. Polynucleotides can have any three-dimensional structure. A nucleic acid can be double-stranded or single-stranded (i.e., a sense strand or an antisense strand). Non-limiting examples of polynucleotides include genes, gene fragments, exons, introns, messenger RNA (mRNA), transfer RNA, ribosomal RNA, siRNA, micro-RNA, ribozymes, cDNA, recombinant polynucleotides, branched polynucleotides, plasmids, vectors, isolated DNA of any sequence, isolated RNA of any sequence, nucleic acid probes, and primers, as well as nucleic acid analogs. The nucleic acid molecules can be synthesized (for example, by phosphoramidite based synthesis) or obtained from a biological cell, such as the cell of a mammal. The nucleic acids can be those of mammal, e.g., humans, a non-human primates, cattle, horses, pigs, sheep, goats, dogs, cats, rabbits, guinea pigs, hamsters, rats, or mice.

An “isolated” nucleic acid can be, for example, a naturally-occurring DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule, independent of other sequences (e.g., a chemically synthesized nucleic acid, or a cDNA or genomic DNA fragment produced by the polymerase chain reaction (PCR) or restriction endonuclease treatment). An isolated nucleic acid also refers to a DNA molecule that is incorporated into a vector, an autonomously replicating plasmid, a virus, or into the genomic DNA of a prokaryote or eukaryote. In addition, an isolated nucleic acid can include an engineered nucleic acid such as a DNA molecule that is part of a hybrid or fusion nucleic acid. A nucleic acid existing among hundreds to millions of other nucleic acids within, for example, cDNA libraries or genomic libraries, or gel slices containing a genomic DNA restriction digest, is not to be considered an isolated nucleic acid.

Isolated nucleic acid molecules can be produced by standard techniques. For example, polymerase chain reaction (PCR) techniques can be used to obtain an isolated nucleic acid containing a nucleotide sequence described herein. PCR can be used to amplify specific sequences from DNA as well as RNA, including sequences from total genomic DNA or total cellular RNA. Various PCR methods are described, for example, in PCR Primer: A Laboratory Manual, Dieffenbach and Dveksler, eds., Cold Spring Harbor Laboratory Press, 1995. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid. Isolated nucleic acids also can be chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated into a vector. Isolated nucleic acids disclosed herein also can be obtained by mutagenesis of, e.g., a naturally occurring DNA.

As used herein, the term “percent sequence identity” refers to the degree of identity between any given query sequence and a subject sequence. A subject sequence typically has a length that is more than 80 percent, e.g., more than 82, 85, 87, 89, 90, 93, 95, 97, 99, 100, 105, 110, 115, or 120 percent, of the length of the query sequence. A query nucleic acid or amino acid sequence can be aligned to one or more subject nucleic acid or amino acid sequences using the computer program ClustalW (version 1.83, default parameters), which allows alignments of nucleic acid or protein sequences to be carried out across their entire length (global alignment). Chema et al. (Nucleic Acids Res. 31(13):3497-500, 2003). ClustalW can be run, for example, at the Baylor College of Medicine Search Launcher site (searchlauncher.bcm.tmc.edu/multi-align/multi-align.html) and at the European Bioinformatics Institute site on the World Wide Web (ebi.ac.uk/clustalw).

The term “exogenous” with respect to a nucleic acid indicates that the nucleic acid is part of a recombinant nucleic acid construct, or is not in its natural environment. For example, an exogenous nucleic acid can be a sequence from one species introduced into another species, i.e., a heterologous nucleic acid. Typically, such an exogenous nucleic acid is introduced into the other species via a recombinant nucleic acid construct. An exogenous nucleic acid can also be a sequence that is native to an organism and that has been reintroduced into cells of that organism. An exogenous nucleic acid that includes a native sequence can often be distinguished from the naturally occurring sequence by the presence of non-natural sequences linked to the exogenous nucleic acid, e.g., non-native regulatory sequences flanking a native sequence in a recombinant nucleic acid construct. In addition, stably transformed exogenous nucleic acids typically are integrated at positions other than the position where the native sequence is found.

It will be appreciated that a number of nucleic acids can encode a polypeptide having a particular amino acid sequence. The degeneracy of the genetic code is well known to the art; i.e., for many amino acids, there is more than one nucleotide triplet that serves as the codon for the amino acid.

A “vector” is a replicon, such as a plasmid, phage, or cosmid, into which another DNA segment may be inserted so as to bring about the replication of the inserted segment. Generally, a vector is capable of replication when associated with the proper control elements. Suitable vector backbones include, for example, those routinely used in the art such as plasmids, viruses, artificial chromosomes, BACs, YACs, or PACs. The term “vector” includes cloning and expression vectors, as well as viral vectors and integrating vectors. An “expression vector” is a vector that includes a regulatory region. Suitable expression vectors include, without limitation, plasmids and viral vectors derived from, for example, bacteriophage, baculoviruses, and retroviruses. Numerous vectors and expression systems are commercially available from such corporations as Novagen (Madison, Wis.), Clontech (Palo Alto, Calif.), Stratagene (La Jolla, Calif.), and Invitrogen/Life Technologies (Carlsbad, Calif.).

Vectors typically contain one or more regulatory regions. The term “regulatory region” refers to nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, and introns.

As used herein, the term “operably linked” refers to positioning of a regulatory region and a sequence to be transcribed in a nucleic acid so as to influence transcription or translation of such a sequence. For example, to bring a coding sequence under the control of a promoter, the translation initiation site of the translational reading frame of the polypeptide is typically positioned between one and about fifty nucleotides downstream of the promoter. A promoter can, however, be positioned as much as about 5,000 nucleotides upstream of the translation initiation site, or about 2,000 nucleotides upstream of the transcription start site. A promoter typically comprises at least a core (basal) promoter. A promoter also may include at least one control element, such as an enhancer sequence, an upstream element or an upstream activation region (UAR). The choice of promoters to be included depends upon several factors, including, but not limited to, efficiency, selectability, inducibility, desired expression level, and cell- or tissue-preferential expression. It is a routine matter for one of skill in the art to modulate the expression of a coding sequence by appropriately selecting and positioning promoters and other regulatory regions relative to the coding sequence.

The vectors also can include, for example, origins of replication, scaffold attachment regions (SARs), and/or markers. A marker gene can confer a selectable phenotype, e.g., antibiotic resistance, on a cell. In addition, an expression vector can include a tag sequence designed to facilitate manipulation or detection (e.g., purification or localization) of the expressed polypeptide. Tag sequences, such as green fluorescent protein (GFP), glutathione S-transferase (GST), polyhistidine, c-myc, hemagglutinin, or Flag™ tag (Kodak, New Haven, Conn.) sequences typically are expressed as a fusion with the encoded polypeptide. Such tags can be inserted anywhere within the polypeptide, including at either the carboxyl or amino terminus.

The expression vectors disclosed herein containing the above described coding can be used, for example, to transfect or transduce either prokaryotic (e.g., bacteria) cells or eukaryotic cells (e.g., yeast, insect, or mammalian) cells. Such cells can then be used, for example, for large or small scale in vitro production of the fibrinogen polypeptides by methods known in the art. In essence, such methods involve culturing the cells under conditions which maximize production of the fusion protein and isolating the fusion protein from the cells or from the culture medium.

Sleeves and Bioreactors: Cells useful in the present compositions can be derived from the intended recipient or an allogeneic donor. Cell types with which the biocompatible tissue repair compositions can be repopulated include, but are not limited to, embryonic stem cells (ESC), adult or embryonic mesenchymal stem cells (MSC), monocytes, hematopoetic stem cells, gingival epithelial cells, endothelial cells, fibroblasts, or periodontal ligament stein cells, prochondroblasts, chondroblasts, chondrocytes, pro-osteoblasts, osteocytes, or osteoclast. Any combination of two or more of these cell types (e.g., two, three, four, five, six, seven, eight, nine, or ten) may be used to repopulate the biocompatible tissue repair composition. Methods for isolating specific cell types are well-known in the art. Donor cells may be used directly after harvest or they can be cultured in vitro using standard tissue culture techniques. Donor cells can be infused or injected into the biocompatible tissue repair composition in situ just prior to placing of the biocompatible tissue repair composition in a mammalian subject. Donor cells can also be cocultured with the biocompatible tissue repair composition using standard tissue culture methods known to those in the art.

As noted, cells useful in the context of the present compositions can be stem cells, for example an embryonic stem cell or an adult stem cell. Adult stem cells can be harvested from many types of adult tissues, including bone marrow, blood, skin, gastrointestinal tract, dental pulp, the retina of the eye, skeletal muscle, liver, pancreas, and brain. The methods are not limited to undifferentiated stem cells and can include those cells that have committed to a partially differentiated state, for example, a mesenchymal stem cell, a hematopoictic stem cell, an endothelial stem cell, or a neuronal stem cell. Such a partially differentiated cell may be precursor to an aclipocyte, an osteocyte, a hepatocyte, a chondrocyte, a neuron, a myocyte, a blood cell, an endothelial cell, an epithelial cell, or a endocrine cell. Established cell lines, for example, embryonic stem cell lines, are also embraced by the methods. Optionally, the cell can have been modified to express one or more exogenous genes (e.g., a gene that expresses a deficient protein or supplies a growth or differentiation factor). The compositions can include cells of mammalian origin (e.g., cells of humans, mice, rats, canines, cows, horses, felines, and ovines), as well as cells from non-mammalian sources.

Cell delivery to tissues (e.g., the myocardium) may be limited due to the presence of a harsh environment (e.g., an infarct). To help overcome this problem, the cells may be heat-shocked prior to implantation. Alternatively or in addition, transfection with a cell survival gene (such as Akt) may be necessary. In addition, there may be a decrease in survival rate for the threads that are further from the perfused/infarcted boundary. An increased angiogenic response may be possible by incorporating growth factors (such as VEGF) into the microthreads. The conditions found in contracting myocardium may require a stronger composite, which could be accomplished, for example, by increasing the number of microthreads used in the composite suture and/or crosslinking the threads.

Therapeutic agents: Therapeutic agents that aid tissue repair or regeneration can be included in the fibrin microthread compositions. These agents can include growth factors including cytokines and interleukins, extracellular matrix proteins and/or biologically active fragments thereof (e.g., RGD-containing peptides), blood and serum proteins, nucleic acids, hormones, vitamins, chemotherapeutics, antibiotics and cells. These agents can be incorporated into the compositions prior to the compositions being placed in the subject. Alternatively, they can be injected into or applied to the composition already in place in a subject. These agents can be administered singly or in combination. For example, a composition can be used to deliver cells, growth factors and small molecule therapeutics concurrently, or to deliver cells plus growth factors, or cells plus small molecule therapeutics, or growth factors plus small molecule therapeutics.

Growth factors that can be incorporated into the biocompatible tissue repair composition include any of a wide range of cell growth factors, angiogenic factors, differentiation factors, cytokines, hormones, and chemokines known in the art. Growth factors can be polypeptides that include the entire amino acid sequence of a growth factor, a peptide that corresponds to only a segment of the amino acid sequence of the native growth factor, or a peptide that derived from the native sequence that retains the bioactive properties of the native growth factor. The growth factor can be a cytokine or interleukin. Any combination of two or more of the factors can be administered to a subject by any of the means recited below. Examples of relevant factors include vascular endothelial cell growth factors (VEGF) (e.g., VEGF A, B, C, D, and E), platelet-derived growth factor (PDGF), insulin-like growth factor (IGF) I and IGF-II, interferons (IFN) (e.g., IFN-α, β, or γ), fibroblast growth factors (FGF) (e.g., FGF1, FGF-2, FGF-3, FGF-4-FGF-10), epidermal growth factor, keratinocyte growth factor, transforming growth factors (TGF) (e.g., TGFα or β), tumor necrosis factor-α, an interleukin (IL) (e.g., IL-1, IL-2, Il-17-IL-18), Osterix, Hedgehogs (e.g., sonic or desert), SOX9, bone morphogenetic proteins (BMP's), in particular, BMP 2, 4, 6, and 7 (BMP-7 is also called OP-1), parathyroid hormone, calcitonin prostaglandins, or ascorbic acid.

Factors that are proteins can also be delivered to a recipient subject by administering to the subject: (a) expression vectors (e.g., plasmids or viral vectors) containing nucleic acid sequences encoding any one or more of the above factors that are proteins; or (b) cells that have been transfected or transduced (stably or transiently) with such expression vectors. Such transfected or transduced cells will preferably be derived from, or histocompatible with, the recipient. However, it is possible that only short exposure to the factor is required and thus histo-incompatible cells can also be used.

Other useful proteins can include, without limitation, hormonse, an extracellular antibodies, extracellular matrix proteins, and/or biologically active fragments thereof (e.g., RGD-containing peptides) or other blood and serum proteins, e.g., fibronectin, albumin, thrombospondin, von Willebrand factor and fibulin.

Naturally, administration of the agents mentioned above can be single, or multiple (e.g., two, three, four, five, six, seven, eight, nine, 10, 15, 20, 25, 30, 35, 40, 50, 60, 80, 90, 100, or as many as needed). Where multiple, the administrations can be at time intervals readily determinable by one skilled in art. Doses of the various substances and factors will vary greatly according to the species, age, weight, size, and sex of the subject and are also readily determinable by a skilled artisan.

Tissue repair: As noted, a wide variety of tissues can be repaired by the present devices, and an exemplary tissue is the myocardium, which may be damaged by numerous types of cardiovascular disease or trauma. For example, in the event of coronary artery disease, a disease of the arteries that supply blood and oxygen to the heart, decreased blood flow to the heart muscle results in regions starved for oxygen and nutrients and consequently damaged. The ischemic tissue treatable as described herein may also result from a heart attack, where a coronary artery becomes suddenly blocked, stopping the flow of blood to the heart muscle and damaging it.

Methods of labeling and tracking stern cells: Traditional tracking agents such as green fluorescent protein (GFP) or fluorescent dyes fail to illuminate delivered cells above high levels of autofluorescence in the heart (Laflamme and Murry Nat. Biotechnol. 23:845-856, 2005). Secondary staining used to detect LacZ or amplify GFP generates false positives and also involves painstaking efforts to identify the exogenous cells in hundreds of tissue sections. More recently, cells have been labeled with inorganic particles for detection by magnetic resonance imaging (MRI) or PET, but these imaging approaches resolve no fewer than thousands of cells. None of the existing tracking techniques offers the ability to unambiguously identify delivered cells in vivo with single-cell resolution using relatively high-throughput approaches (i.e., no secondary staining). In order to follow the fate of the hMSCs delivered to the myocardium, we developed an approach using intracellular quantum dots (QDs; highly fluorescent nanoparticles possessing unique optical properties) (Rosen et al, Stem Cells 2007). Accordingly, methods of labeling stem cells (e.g., hMSCs) as described below and the cells so labeled are within the scope of the present invention.

Human MSCs were incubated in QD solution (8.2 nM solution of 655 ITK Carboxyl QDs in Cambrex MSCGM) for 24 hours at 37° C. This provided clear demarcation of the hMSCs with QDs found in the cytoplasm (FIG. 2). Cells were subsequently analyzed using a LSR II true multiparameter flow cytometer analyzer and greater than 96% of four sets of QD loaded hMSCs (each containing a minimum of 17,000 cells) were positive for QDs. A number of additional experiments were performed and demonstrated that QDs can be detected up to 8 weeks in vivo (FIG. 2B), are not taken up by cardiac myocytes in vitro or in vivo and do not affect hMSC proliferation or differentiation (FIG. 2C) (Rosen et al., supra). Other features, objects, and advantages of the invention will be apparent from the description and drawings, and from the claims.

### Example 1

### Materials and Methods for Making Fibrin Microthreads

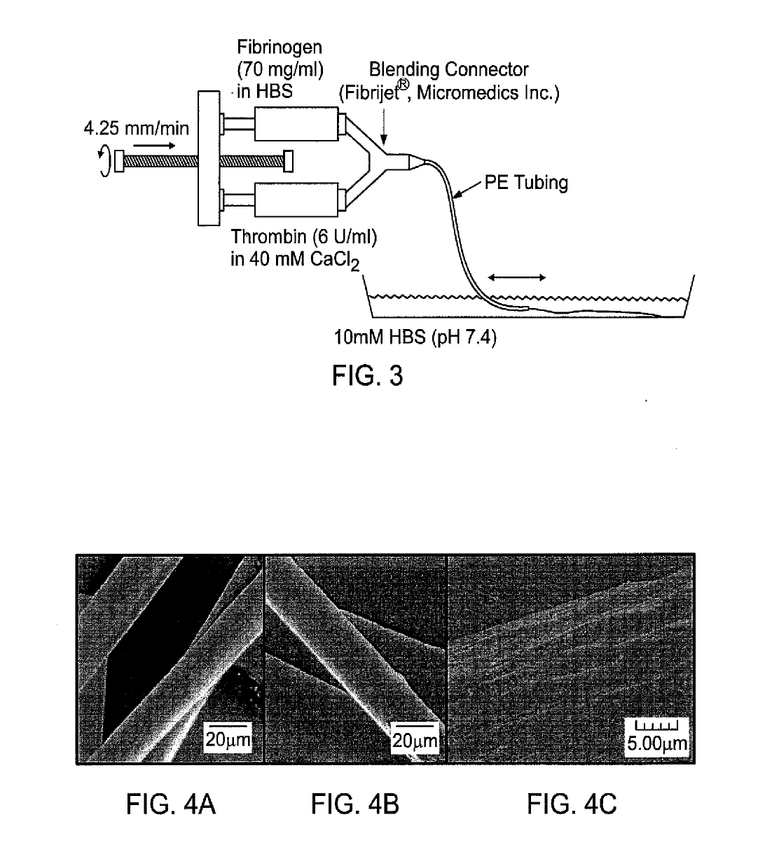

Fibrin microthread preparation: Fibrin microthreads were co-extruded from solutions of fibrinogen and thrombin according to the schematic shown in FIG. 3 of the attached Example 7. Fibrinogen from bovine plasma (Sigma, St. Louis, Mo., catalogue number F4753) was dissolved in HEPES Buffered Saline (HBS, 20 mM HEPES, 0.9% NaCl) at 70 mg/mL and stored at −20° C. Thrombin from bovine plasma (Sigma, St. Louis, Mo., catalogue number T4648) was stored frozen as a stock solution at a concentration of 40 U/mL in FIBS. A working solution of thrombin was diluted from the stock to a final concentration of 6 U/mL in a 40 mM CaCl₂ solution. Both the fibrinogen and thrombin solutions were warmed to 37° C. and placed into separate 1 mL syringes. The solutions were coextruded using a stabilized crosshead on a threaded rod with a crosshead speed of 4.25 mm/min through a blending applicator tip (Micromedics, Inc., St. Paul, Minn.). The blending applicators were Luer locked to the two syringes through individual bores and mixed in a needle that was Luer locked to the tip. The solutions were combined and extruded through polyethylene tubing (BD, Sparks, Md.) with an inner diameter of 0.38 mm into a bath of 10 mM HEPES, pH 7.4 at room temperature. The threads were hand-drawn through the bath at a rate approximately matching the flow rate of the polymerization solution form the tubing. The bath was contained in a vessel that had a Teflon®coated surface. Finally, threads were removed from the bath, air dried under the tension of their own weight, and stored at room temperature in a desiccator until use.

Fibrin microthread crosslinking. In the event additional strength is required, any of the biopolymers described herein can be additionally crosslinked. Here, microthreads were crosslinked by UV irradiation. Microthreads were placed on a reflective aluminum foil surface that was centered 11 cm from a bank of 5-8 watt UV tubes emitting at a primary wavelength of 254 nm in a model CL-1000 ultraviolet crosslinker (UVP, Upland, Calif.). The microthreads were exposed for 0, 20, 40, 60, and 120 minutes and therefore received a calculated total energy of 8.5, 17.1, 25.7, 51.3 J/cm². Controls were left uncrosslinked (0 J/cm²).

Scanning Electron Microscopy (SEM). Fibrin microthreads were imaged with a scanning electron microscope to characterize thread morphology and surface topography. Air dried fibrin threads were mounted on aluminum stubs (Ted Pella, Inc., Redding, Calif.) coated with double-sided carbon tape and sputtered coated with a thin layer of gold-palladium for 2 minutes. Images were acquired at 15 kV using a JSM-KLG scanning electron microscope.

Thread Swelling. Qualitative volumetric analyses were based on the swelling ratios of fibrin microthreads. The cross-sectional area of each thread was calculated from an average of three diameter measurements along its length, assuming cylindrical thread geometry. The diameters were measured both dry and after hydration for at least 30 minutes in phosphate buffered saline (PBS) using a 20× objective on a Nikon Eclipse E400 microscope fitted with a calibrated reticule. The swelling ratio was calculated as the ratio of the wet cross-sectional area to the dry cross-sectional area for each discrete thread.

Mechanical Properties. Fibrin microthreads were hydrated and mechanically loaded in uniaxial tension to obtain stress-strain curves. Individual threads were mounted vertically with adhesive (Silastic Silicone Type A, Dow Corning) on vellum frames with precut windows that defined the region of loading. For tensile testing, the samples in the vellum frames were clamped into a custom designed micromechanical testing unit consisting of a horizontal linearly actuated crosshead and a fixed 150 g load cell. An initial gauge length of 20 mm was defined as the distance between adhesive spots across the precut window in the vellum frame. Test unit operations and data acquisition were controlled with LabView software (National Instruments, Austin, Tex.). Threads were hydrated for at least 30 minutes prior to testing, but were not tested submerged. After loading into the testing apparatus, the edges of each frame were cut leaving the thread intact. The threads were then loaded to failure at a 50% strain rate (10 mm/min). Curves of the Piola Kirchhoff stress versus Green's strain were calculated from the load displacement data assuming a cylindrical cross-sectional area of each thread and calculating cross-sectional area based on thread diameter measurements as described above for swelling ratio. Post-processing of the mechanical data considered a strain of zero to be when a thread was minimally loaded to a nominal threshold of 0.01 grams, or less than 1% of the ultimate load for the weakest uncrosslinked thread. Ultimate tensile strengths (UTS), strains at failure (SAF), and the maximum tangent moduli or stiffnesses (E) were calculated from the stress-strain curves. The stiffness was defined as the maximum value for a tangent to the stress-strain curve over an incremental strain of 0.03.

Cell Proliferation. Normal human dermal fibroblasts were isolated from neonatal foreskins. Foreskins were trimmed with scissors to remove excess fatty tissue, rinsed repeatedly with sterile phosphate-buffered saline, and diced into small fragments. The fragments were allowed to adhere to the bottom of a tissue culture plate in a humidified 10% CO₂ atmosphere at 37° C. for 1 hour, and were then covered with Dulbecco's modified Eagle's medium (DMEM; high glucose, Gibco BRL, Gaithersburg, Md.) supplemented with 20% fetal bovine serum (PBS; JRH Biosciences, Lenexa, Kans.) containing 100 U of penicillin and 100 μg of streptomycin (Gibco BRL) per ml. Over a period of 14 days, fibroblasts migrated from the tissue fragments and formed a confluent layer on the tissue culture plate. Fibroblasts were cultured in Dulbecco's Modified Eagle's Medium (DMEM; Gibco BRL, Gaithersburg, Md.) supplemented with 10% fetal bovine serum (FBS; Atlanta Biologicals, Lawrenceville, Ga.) and penicillin/streptomycin (100 U/100 mg per mL; Gibco BRL) in an incubated chamber maintained at 37° C. and 10% CO₂. Passages 4-7 were used during experiments.

To characterize cell attachment and proliferation, bundles of 10 fibrin threads 1.5 cm long, (uncrosslinked fibrin, UV crosslinked fibrin (40 minutes), or polypropylene controls (Prolene 7-0 suture)) were glued to Thermanoxml coverslips (Nalge Nunc International, Rochester, N.Y.) with silicone adhesive (Silastic Silicone Type A, Dow Corning) and placed individually inside standard 35 mm culture dishes. Thread bundles were rehydrated in PBS for 15 minutes, sterilized with 70% isopropyl alcohol for 1 hour and rinsed in sterile PBS for 15 minutes, 3 times. Following standard procedure for passaging, fibroblasts were released from monolayer culture with trypsin, centrifuged, and resuspended at a concentration of 500,000 cells/mL. Each sterilized thread bundle was seeded with 100 μL of cells in media with 10% FBS and incubated for 30 minutes. Two mL of media were then added to each culture dish and returned to incubation conditions. Fibroblast attachment and proliferation was visualized at days 1 and 7 with a Live/Dead cell viability stain (Molecular Probes, Eugene, Oreg.). At each time point, after removal of media, 1.5 mL of a 4 μM ethidium homodimer-1 and 2 μM calcein AM solution were added to each bundle of threads and incubated at room temperature. Calcein (green, Ex/em 495 nm/515 nm) is retained in living cells while ethidium (red, Ex/em 495 nm/635 nm) is excluded by intact plasma membranes, but enters damaged membranes where it can fluoresce upon binding to nucleic acid. Thread bundles were cut from Thermanoxml coverslips and placed on slides for fluorescent imaging. Images were acquired on a Nikon Eclipse E400 microscope using a Texas Red filter cube.

Statistical Analyses. Statistical differences between means of the data were conducted by one-way ANOVA with pairwise multiple comparisons (Holm-Sidak method) using SigmaStat (Systat Software Inc., Point Richmond, Calif.). Values reported are means and standard deviations unless otherwise stated. A p<0.009 indicated a significant difference between experimental groups.

### Example 2

### Synthesis of Microthreads and Analysis of Coextrusion Parameters

Biodegradable microthreads were synthesized from collagen or fibrin. Acid soluble type I collagen was obtained from rat tails following a procedure outlined by Cornwell K G, et al. Collagen solutions (10 mg/ml in 5 mM HCl) were extruded through 0.38 mm inner diameter polyethylene tubing (Becton Dickinson, Inc., Franklin, N.J.) using a syringe pump (KD Scientific, New Hope, Pa.) set at a flow rate of 0.7 mL/minute. Threads were extruded into a bath of fiber formation buffer (pH 7.4, 135 mM NaCl, 30 mM TrizmaBase, and 5 mM NaPO₄ dibasic) and maintained overnight. The buffer was then replaced with fiber incubation buffer (pH 7.4, 135 mM NaCl, 10 mM TrizmaBase, and 30 mM NaPO₄ dibasic) that was maintained at 37° C. overnight. The incubation buffer was then replaced with distilled water, and maintained at 37° C. overnight. The threads were removed from the water bath and air-dried for future use in experimental studies.

In other studies, fibrin microthreads were coextruded from solutions of fibrinogen and thrombin using techniques developed by Cornwell and Pins. Briefly, fibrinogen (from bovine plasma; Sigma, St. Louis, Mo.; MO F4753) was dissolved in HEPES buffered saline (1-IBS, 20 mM HEPES, 0.9% NaCl) at a final protein concentration of 45.5 mg/mL and thrombin (from bovine plasma; Sigma, St. Louis, Mo.; MO T4648) was diluted to 6 U/mL in 40 mM CaCh solution. Both fibrinogen and thrombin were warmed to 37° C. and aspirated into separate 1 mL syringes. The solutions were coextruded using a stabilized crosshead on a threaded rod through a blending applicator (Micromedics, St. Paul, Minn.) at a speed of 4.25 mm/minute, through polyethylene tubing 0.38 mm in diameter (FIG. 3). The materials were coextruded into a bath of 10 mM HEPES, pH 7.4, at room temperature for an hour. Within 5 minutes, threads formed, largely at the bottom of the bath. Fibrin threads were then removed from the bath, air-dried, and stored at room temperature.

To characterize the structure and morphology of collagen and fibrin threads, scaffolds were analyzed with light and scanning electron microscopy (SEM) techniques following extrusion. SEM analyses indicated that collagen microthreads exhibited cylindrical geometries with slightly rougher surface textures, consistent with the fibril substructure that is characteristic of self-assembled collagen fibers.⁵¹ (FIG. 4) The dry diameters of the collagen microthreads ranged from 48 to 70 μm with an average of 60 μm. Fibrin microthreads were produced with properties comparable to collagen microthreads. Upon air drying, the threads elongated considerably under their own weight, stretching in length while decreasing in initial cross-sectional area. After drying, all fibrin threads appeared to exhibit gross structural and morphological properties comparable to collagen microthreads. The dry diameters of the microthreads ranged from 20 to 50 μm with an average of 34.6 μm and a median of 35 μm. SEM analyses indicated that the fibrin threads had relatively smooth surfaces with regular, submicron surface topographies (FIG. 4). Upon rehydration in PBS, uncrosslinked fibrin threads-swelled to more than four times their dry cross-sectional area.

The effect of coextrusion rate, and pH and temperature of the aqueous bath on fibrin microthread tensile properties was analyzed. Coextrusion rate was expressed as a “rate ratio”, i.e., the ratio of flow velocity/plotter velocity, where flow velocity is the speed with which the fibrin solution emerges from the tubing and plotter velocity is the speed of the extrusion tubing through the aqueous bath. For example, a rate ratio of 2.0 describes extrusion parameters in which the solution flows out of the tubing twice as fast as the tubing tip moves through the aqueous bath. Fibrinogen and thrombin solutions were prepared according to the method in Example 1 and coextruded with rate ratios of either 1.0, 2.0, or 4.0, and analyzed for tensile strength according to the method in Example 1. Increasing the rate ratio from 1.0 to 2.0 resulted in a three-fold increase in ultimate tensile strength and about a ten-fold increase in load to failure. A further increase from 2.0 to 4.0 resulted in a decrease in ultimate tensile strength, but had minimal effect on load to failure. The ultimate tensile strength averaged 4.78 MPa for a rate ratio of 2.0, while ratios above and below generated in fibrin microthreads with statistically significantly lower tensile strength. The load to failure for rate ratios of 2.0 and 4.0 were roughly similar and both were greater than that obtained for the rate ratio of 1.0. Increasing the rate ratio increased both the wet diameter and the strain to failure in a roughly linear fashion.

The effect of pH of the aqueous bath on fibrin microthread tensile strength was also analyzed. Fibrinogen and thrombin solutions were prepared according to the method in Example 1 and coextruded into solutions of 10 mM HEPES-buffered saline at either pH 6.0, 7.42, or 8.5. At physiological pH (7.42) and higher (8.5) the ultimate tensile strength of the resulting fibrin microthread was about seven- and five-fold greater, respectively than that of fibrin microthreads formed at pH 6.0.

The effect of the temperature of the aqueous bath on fibrin microthread tensile strength was also analyzed. Fibrinogen and thrombin solutions were prepared according to the method in Example 1 and coextruded into a solution of 10 mM HEPES-buffered 7.42 at either 20° C. or 37° C. The ultimate tensile strength of the fibrin microthreads formed at 20° C. was statistically significantly greater than those produced at 37° C.

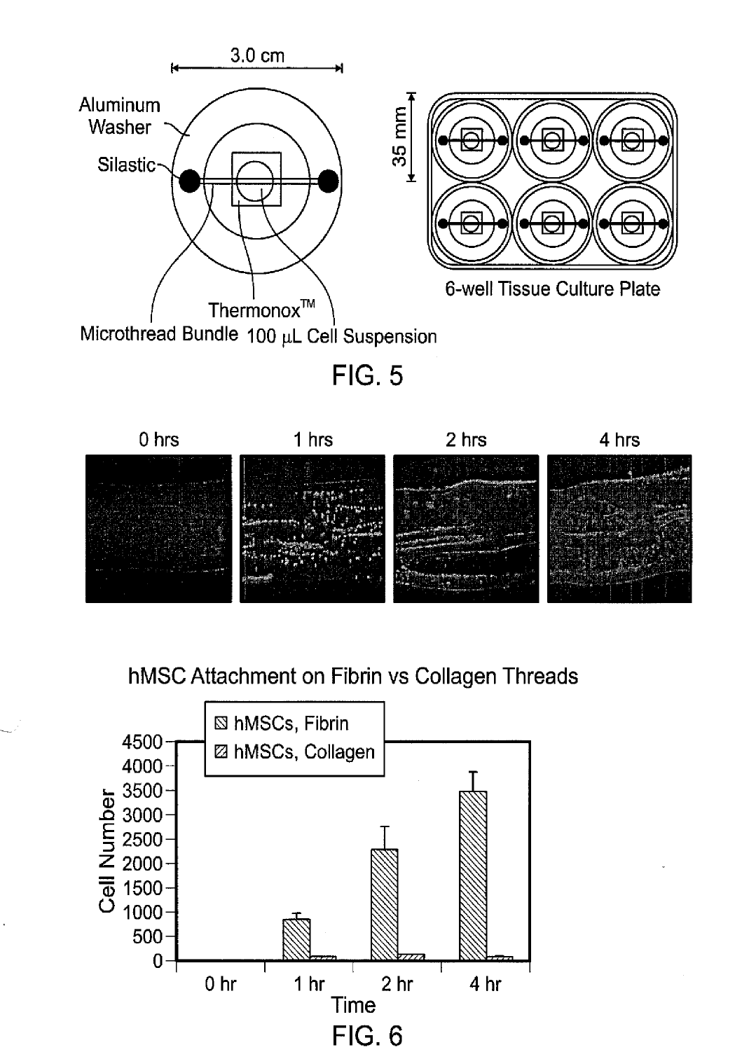

To determine if hMSCs attach to type I collagen threads or fibrin threads, we visualized hMSCs seeded on threads labeled with Hoechst nuclear staining and cytoplasm-loaded Quantum-Dots. First, individual threads were bundled into groups of 10 threads and cut to 2.5 cm in length. The bundles were glued to 3.0 cm outer diameter aluminum washers with silicone adhesive (Silastic Silicone Type A, Dow Corning). The aluminum washers fit into the 35 mm wells of a 6-well tissue culture plate (Becton Dickinson, Franklin Lakes, N.J.). Before the washers with threads are placed into the wells, Thermanox™ coverslips (Nalge Nunc International, Rochester, N.Y.) are glued with the same silicone adhesive to the middle of each well to serve as defined cell-seeding areas. The threads on the washer are rehydrated in PBS for 15 minutes, sterilized with 70% isopropyl alcohol for 1 hour, and then rinsed three times in sterile PBS for 15 minutes. Once sterilized, the threads on washers are placed on top of the Thermanox™ coverslip in the 35 mm well. Following standard procedure for passaging, Quantum-Dot loaded hMSCs (described above) are released from monolayer with trypsin, centrifuged, and resuspended at a concentration of 500,000 cells/mL in 10% FBS in DMEM. 100 μL of hMSC suspension are added to each well, over the threads and onto the Thermanox™ coverslip (FIG. 5). The 6-well tissue culture plates are then be placed into 37° C., 5% CO₂ incubators. The threads are removed from the 35 mm wells and washed twice with sterile PBS for 5 minutes. The threads are then stained with Hoechst dye (Cambrex Bio Science, Walkcrsville, Md.), applied to the threads for 10 minutes, and then washed once with PBS for 5 minutes. The thread bundles are then removed from the washers, placed on a glass slide, and viewed under a fluorescent microscope.

After 4 hours of incubation time, hMSC showed a time dependent linear attachment rate to fibrin threads. After 4 hours of incubation, approximately 400 cells/mm of thread bundle length were found. In addition, hMSCs more readily adhered to fibrin threads compared to collagen threads (FIG. 6).

### Example 3

### Fibrin Microthread Structure and Morphology

The structure and morphology of fibrin microthreads were analyzed with light and scanning electron microscopy techniques. The transparent solutions of fibrinogen and thrombin were co-extruded into the bath. Within 5 minutes threads formed, largely at the bottom of the bath. Upon removal from the buffer and air drying, the threads elongated considerably under their own weight, stretching in length while decreasing in initial cross-sectional area. After drying, all fibrin threads visually appeared to have relatively consistent gross structure and morphology that remained unchanged after crosslinking. The dry diameters of the microthreads ranged from 20 to 50 μm with an average of 34.6 μm and a median of 35 μm. SEM analyses indicated that the fibrin threads had relatively smooth surfaces with regular, submicron surface topographies. Upon rehydration in PBS, uncrosslinked fibrin threads swelled to more than 4 times their dry cross-sectional areas (Table 1). In contrast, threads that were crosslinked with UV light swelled significantly less than uncrosslinked threads, achieving swelling ratios that peaked at approximately 2.5 and decreased slightly with increased exposure times.

TABLE 1
The cross-sectional area and swelling ratio of fibrin
microthreads with increased UV cross-linking

| UV | | Sample | | | |
|---|---|---|---|---|---|
| Exposure | Power | Size | Dry Area | Hydrated | Swelling |
| time (min) | (J/cm2) | (n) | (uM) | Area (uM) | Ratio |
| 0 | 0.00 | 13 | 910 ± 400 | 3200 ± 1670 | 4.09 ± 1.48 |
| 20 | 8.55 | 19 | 1210 ± 560 | 2950 ± 1550 | 2.59 ± 0.66 |
| 40 | 17.10 | 18 | 1070 ± 410 | 2490 ± 1020 | 2.42 ± 0.65 |
| 60 | 25.66 | 18 | 1210 ± 570 | 2820 ± 1440 | 2.38 ± 0.57 |
| 120 | 51.31 | 12 | 940 ± 250 | 1890 ± 820 | 2.24 ± 0.44 |

### Example 4

### Fibrin Microthread Mechanical Properties

The mean ultimate tensile strengths (UTS), failure strains, and moduli of mechanically tested discrete fibrin microthreads are summarized in Table 2. In general, fibrin threads exhibited extended initial toe regions of increasing elongation with little increase in stress follow by a rapid ascension in stress until failure. Uncrosslinked threads attained average UTS of 4.48 MPa, typically breaking at strains of less than one-third of the original lengths of the threads. The UTS of the threads increased with UV exposure. The maximal strengths were achieved when threads were exposed to 17.10 J/cm² of UV light. The strengths measured at this exposure level were significantly greater than other conditions tested in this study. While the strains to failure exhibited a small declining trend with increased UV exposure, the decrease was nominal and not significantly different. The modulus, measured as the maximum tangent modulus over an incremental strain of 0.03, established a similar trend to UTS. This measure or the bulk material stiffness increased with UV exposure before reaching a plateau when threads were treated with 17.10 J/cm² of UV energy.

TABLE 2
The mechanical properties of fibrin microthreads
with increased UV cross-linking

| UV Exposure | Power | Sample Size | Strength | Failure | Modulus, E |
|---|---|---|---|---|---|
| time (min) | (J/cm2) | (n) | UTS (MPa) | Strain, SAF | (MPa) |
| 0 | 0.00 | 22 | 4.48 ± 1.79 | 0.31 ± 0.15 | 60.70 ± 25.71 |
| 20 | 8.55 | 19 | 5.29 ± 2.78 | 0.26 ± 0.13 | 88.54 ± 27.53 |
| 40 | 17.10 | 19 | 7.82 ± 3.10 | 0.27 ± 0.08 | 111.39 ± 67.48 |
| 60 | 25.66 | 19 | 6.58 ± 3.03 | 0.25 ± 0.11 | 103.89 ± 53.47 |
| 120 | 51.31 | 11 | 5.88 ± 3.45 | 0.19 ± 0.12 | 81.41 ± 66.90 |

### Example 5

### Fibroblast Attachment and Proliferation

The attachment and proliferation of fibroblasts to bundles of fibrin threads were evaluated qualitatively at days 1 and 7 for the investigation of biocompatibility and the support of cell growth for applications in tissue regeneration. One day after cell seeding, fibroblasts attached readily to both the uncrosslinked and UV crosslinked fibrin threads as visualized with a viability stain. Furthermore, both supported more fibroblast attachment than polypropylene threads. On all three thread types, fibroblasts tended to align along the long axis of the threads and in the grooves between threads in the bundles. While most cells were viable, non-viable cells were occasionally visualized on all thread types. By 7 days, viable cells were visualized on all thread types including controls. However, while areas of the crosslinked fibrin threads maintained relatively constant viable cell quantities compared to day 1, uncrosslinked threads supported robust proliferation. Fibroblasts on uncrosslinked fibrin threads were completely confluent with sheets of cells spanning the length of the threads and filling gaps between threads. While non-viable cells could be distinguished on all thread types, UV crosslinked fibrin threads fluoresced moderately in the red wavelengths, making non-viable cells more difficult to view and image.

Cell seeding is illustrated in FIG. 8.

### Example 6

### Effect of Fibroblast Growth Factor-2 (FGF-2) on Fibroblast Attachment and Proliferation on Fibrin Microthreads

The effect of FGF-2 on fibroblast attachment and proliferation on fibrin microthreads was analyzed in two ways. In the first method, soluble FGF-2 was added to cells cultured on fibrin microthreads. Fibroblasts were seeded on fibrin microthreads in serum-free medium according to the method described in Example 1, in the presence or absence of 100 ng/mL of FGF-2. Media was changed daily over a period of seven days. The mean migration distance on day 7 was statistically significantly greater than that observed in the absence of soluble FGF-2. In the second method, FGF-2 was incorporated into fibrin microthreads during synthesis. Fibrin microthreads were prepared according to the method in Example 1, except that FGF-2 was added to the fibrinogen solution at a final concentration of 25, 50, 100 or 200 ng/mL. Cells were seeded according to the method described in Example 1 and tissue ingrowth rate (mm/day) and total cell numbers were measured over a period of seven days.

### Example 7

### Microthreads Sutured into Myocardium Ex Vivo

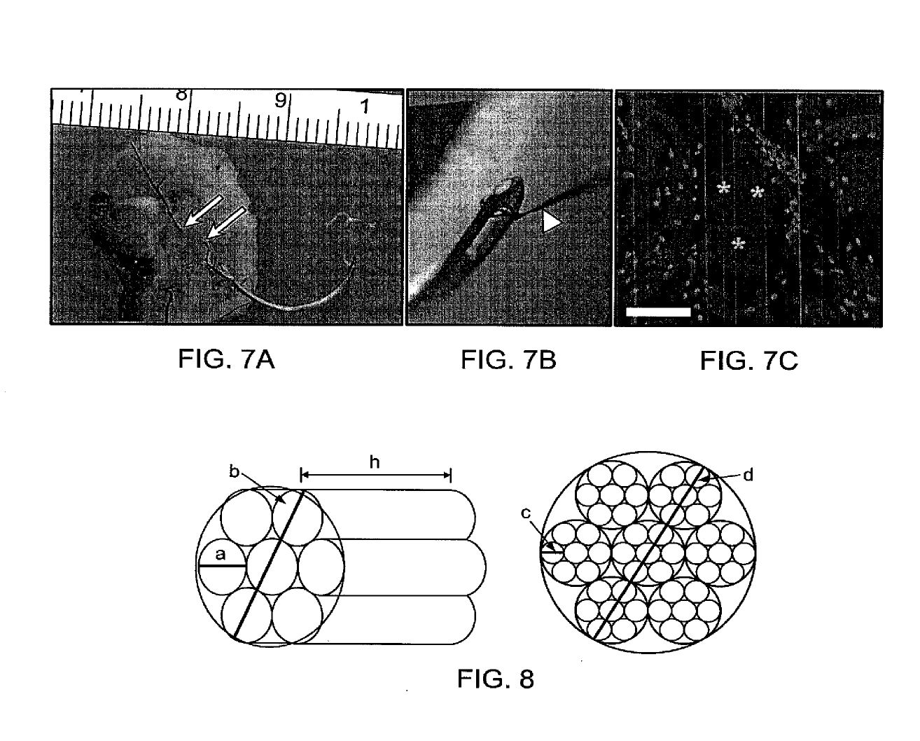

To evaluate whether or not the fibrin microthreads possess the mechanical strength to withstand implantation, microthreads were threaded through the eye of a curved stainless steel surgical needle and sutured into a piece of myocardium ex vivo (FIG. 7). Microthreads were incubated in 10% Trypan blue dye for 20 minutes to improve gross visualization. Canine myocardium, previously fixed in paraformaldehyde, was used as model myocardial tissue for these initial studies. The microthreads were easily pulled through the myocardium and showed no signs of mechanical failure.

To examine the morphology of fibrin microthreads implanted in the heart, a bundle of three microthreads (not stained with Trypan blue) was similarly threaded through a surgical needle and sutured into fixed canine myocardium. The tissue was embedded in freezing medium, cryosectioned and counterstained with Hoechst 33342 dye to visualize cell nuclei and tissue morphology (by overexposing the images to increase background fluorescence). Thread bundles did not break during suturing and retained their bundled structure when implanted. These studies provide evidence that fibrin microthreads are strong enough to be utilized as carriers for hMSC delivery to the myocardium.

### Example 8

### Prophetic Examples and Further Analysis

Seeding of hMSCs on Microthreads: Fibrin microthreads will be made in our co-extrusion system. Single threads or thread bundles (up to 10 threads) will be anchored to a guide wire and threaded into a gas permeable tube (Silastic® Laboratory Tubing, Dow Corning). Quantum dot loaded hMSCs (Lonza Biopharmaceuticals, Basel, Switzerland) in media (MSCGM, Lonza Biopharmaceuticals, Basel, Switzerland) will then be infused into the tube and the tube will be sealed. The thread will be incubated in the tube bioreactor for 1-7 days.

After the incubation period, cell viability will be determined using the LIVE/DEAD Viability/Cytoxicity Kit for mammalian cells (Invitrogen Molecular Probes L-3244). The number of cells (per mm of thread length) will be determined based on the quantum dot label and Hoechst 33342 nuclear staining. Some threads will be subjected to trypsinization to remove the hMSCs. To confirm these cells maintain their sternness, cells will be exposed to standard differentiation protocols using adipogenic and osteogenic kits available through Lonza (Adipogenic Differentiation Medium, PT-3004; Osteogenic Differentiation Medium, PT-3002). Human MSCs cultured under normal conditions will serve as a control. For adipogenesis, cells are plated at 2×10⁴ cells per cm² tissue culture surface area and fed every 2-3 days with MSCGM until cultures reached 100% confluence (5-13 days). Cells are fed on the following regimen for a total of three cycles: 3 days with supplemented Adipogenic Induction Medium followed by 1-3 days with Adipogenic Maintenance Medium. Control hMSCs are fed with Adipogenic Maintenance Medium at all times. After the three cycles, all cells are cultured for another week in Adipogenic Maintenance Medium. Cells will be analyzed using light microscopy for characteristic lipid vacuole formation. We will use previously developed MATLAB (MathWorks, Natick, Mass.) algorithms to determine percentage of images occupied by adipocytes. For osteogenesis, cells are plated at 3×10³ cells per cm² tissue culture surface area and cultured overnight in MSCGM. Cells are then fed with Osteogenesis Induction Medium with replacement medium every 3-4 days for 2-3 weeks. Control cells are fed with MSCGM on the same schedule. Cells are analyzed using light microscopy for characteristic cobblestone appearance. In a separate set of cells, flow cytometry (a LSR H true multiparamcter flow cytometer analyzer with custom 655-nm filter; BD Biosciences, San Diego) will be used to analyze the expression of CD73 and CD 105, two markers previously used to evaluate differentiation potential in hMSCs (Simpson et al., supra).

Determining Mechanical Strength of Microthreads: Mechanical testing of cell-seeded microthreads will be performed as previously described (Cornwell and Pins, J. Biomed. Mater. Res. 82A:104-112, 2007). Briefly, microthreads will be hydrated and mechanically loaded in uniaxial tension to obtain stress-strain curves. Individual threads will be mounted vertically with adhesive (Silastic Silicone Type A, Dow Corning) on vellum frames with precut windows that define the region of loading. The samples in the vellum frames will be clamped into a custom-designed micromechanical testing unit consisting of a horizontal linearly actuated crosshead and a fixed 150 g load cell. An initial gauge length of 20 mm is defined as the distance between adhesive spots across the precut window in the vellum frame. Test unit operations and data acquisition are controlled with LabView software (National Instruments, Austin, Tex.). Threads are hydrated for at least 30 minutes prior to testing, but are not tested submerged. After loading into the testing apparatus, the edges of each frame will be cut, leaving the thread intact. The threads will then be then loaded to failure at a 50% strain rate (10 mm/min). Curves of the 1st Piola Kirchhoff stress versus Green's Strain can be calculated from the load displacement data assuming a cylindrical cross-sectional area of each thread and calculating cross-sectional area based on thread diameter measurements. Postprocessing of the mechanical data will define a strain of zero to be when a thread is minimally loaded to a nominal threshold of 0.01 g, or less than 1% of the ultimate load for the weakest thread. Ultimate tensile strength (UTS), strain at fitilure (SAF), and the maximum tangent modulus or stiffness (E) will be calculated from the stress-strain curves. The stiffness will be defined as the maximum value for a tangent to the stress-strain curve over an incremental strain of 0.03. Based on our ability to implant a bundle of three threads into the myocardium, these threads have sufficient UTS. Therefore, the minimum load is 3 times the UTS of an individual fibrin microthread, with a factor of safety of 2.5, resulting in a value of 67.9 MPa.

Regional and Global Function: To assess the function of the whole left ventricle (global function), animals will be anesthetized with ketamine/xylazine intraperitoneally, maintained under anesthesia with isoflurane and the heart will be exposed as described above. Sonomicrometry transducers will be implanted into the heart to determine the volume of the left ventricle. The vena cava will be slowly occluded over 15 seconds to produce a change in preload (end diastolic volume). The relationship of the stroke work to the end diastolic volume (preload recruitable stroke work), which is heart rate and afterload independent, will be used to assess global ventricular function.

Regional systolic function will be assessed using High Density Mapping (HDM; a method developed by the PI to specifically study mechanical function in small regions of the heart) to determine regional stroke work in the infarct region. In order to determine regional function with HDM, a region of interest is defined from the acquired image (FIG. 9). This region of interest is then divided into subimages. The displacement of each subimage is determined between two images by applying a Fourier transform to each subimage, then combining them through an interference function and applying an inverse Fourier transform to the resultant spectrum. This results in an impulse function, which resides at coordinates (u,v) that define the displacement of the subimage. Through this algorithm, displacement can be determined at hundreds of locations within a typical region of interest. Regional stroke work and systolic shortening have conventionally been used to determine regional function in the beating heart. As we are able to determine displacement with a resolution of 500 μm, regional stroke work (and systolic contraction) can be determined in very small regions. This can be accomplished by determining the change in area between four neighboring points. In general, instead of constructing a work loop out of every four neighboring points, the average change in a subregion consisting of 16-25 different areas is used. This enables us to determine function in regions of less than 10 mm², whereas with sonomicrometry function is generally determined in an area greater than 100 mm². We have used this technique to determine regional function in the isolated rabbit heart and the in vivo canine and porcine heart.

Histological Preparation and Assessment of Engraftment: After functional analysis is performed, hearts will be excised, rinsed in isotonic saline, perfusion-fixed in 4% PFA for 24 hours, cryopreserved in 30% sucrose for an additional 24 hours, embedded in freezing matrix (Jung tissue embedding matrix; Leica, Heerbrugg, Switzerland) and stored at −20° C. The number of cells delivered to the heart and the area of engraftment will be analyzed based on QD fluorescence of unstained serial cryosections of the excised hearts. Three-dimensional reconstruction of hMSC graft size will be performed based on QD fluorescence to determine the size of the graft and the distribution of cells within the heart. To verify that the QD signal is due to the presence of the delivered hMSCs, human cells will be identified by in situ hybridization using a human sequence-specific pan-centromeric probe as described previously.

Morphometric Assessment of Myocardial Infarct Dimensions: To evaluate the effects of hMSC delivery on cardiac morphology and infarct size, serial cryoscctions will be stained with hematoxylin and eosin and measurements will be performed as previously described. Briefly, imaging software (Scion Corporation) will be calibrated and used to trace cross-sectional area of the ventricular (LV) wall and lumen, the infarct zone, as well as septal wall and scar thickness. Prior to hematoxylin and eosin staining, fluorescent images will be taken of the same heart sections and QD-positive area and LV wall cross-sectional area will be similarly measured. Graft size and infarct size will be quantified as a percentage of LV cross-sectional area for each heart, and infarct expansion will be calculated as septal thickness/scar thickness×chamber area/LV area.

TUNEL (terminal deoxynucleotidyl transferase dUTP nick end-labeling) Assay: DNA fragmentation will be assessed 1 and 7 days post implantation as an indicator of hMSC apoptosis using a TUNEL staining kit (Boehringer Mannheim). Briefly, tissue sections will be pretreated with 0.2% Triton X-100 in PBS for 30 minutes, followed by proteinase K digestion (20 μg/ml in 10 μM Tris-HCl, pH 7.4 at 37° C. for 20 minutes). TUNEL staining will be performed according to the manufacturer's instructions, and sections will be counterstained with Hoechst 33342. TUNEL-positive (green fluorescence) and QD-positive cells will be counted and expressed as a percentage of the total number of QD-positive cells.

BrdU Incorporation and Assessment of Proliferation: Proliferating hMSCs will be labeled by intraperitoneal injection of 5-bromodeoxyuridine (BrdU, Invitrogen B23151; 10 mg/ml in PBS; 1.0 mL injection per animal) one hour prior to euthanasia. To identify BrdU-positive hMSCs, cryosections will be treated for 3 minutes with pepsin (Sigma P-7000; 0.1 mg/ml in 0.01 N HCl) at 37° C., immersed in 1.5 N HCl for 15 minutes at 37° C. for antigen retrieval, then neutralized by washing twice with 0.1 M borax, pH 8.5. Sections will be rinsed in PBS, then blocked with 1.5% normal rabbit serum in PBS and incubated overnight with a Alexa488-conjugated anti-BrdU antibody (1:200; Invitrogen MD5420), and counterstained with Hoechst 33342 dye (Invitrogen). BrdU-positive, QD-positive cells will be counted and expressed as a percentage of the total QD-positive cells.

What is claimed is:

1. A composition comprising a polymer configured as a plurality of threads, each having a leading end and a trailing end, wherein the threads are encased along at least a portion of their length by an open-ended sleeve.

2. The composition of claim 1, further comprising a plurality of biological cells in association with the threads and, optionally, a therapeutic agent.

3. The composition of claim 1, wherein the leading ends of the threads are attached to a needle.

4. The composition of claim 1, wherein the polymer configured as a plurality of threads comprises a naturally occurring polymer.

5-6. (canceled)

7. The composition of claim 1, wherein the polymer configured as a plurality of threads comprises a synthetic polymer.

8-9. (canceled)

10. The composition of claim 1, wherein the microthreads are braided, bundled or tied to form filaments.

11. (canceled)

12. The composition of claim 2, wherein the cell is a differentiated cell.

13. (canceled)

14. The composition of claim 2, wherein the cell is a stem cell, a precursor cell, or a progenitor cell.

15-16. (canceled)

17. The composition of claim 1, wherein the sleeve is gas-permeable.

18. The composition of claim 1, wherein the sleeve comprises a synthetic polymer, a natural polymer or a combination thereof.

19. The composition of claim 2, wherein the therapeutic agent is a growth factor, a protein, a vitamin, a mineral, an antimicrobial agent, or a small organic molecule.

20. (canceled)

21. A method of preparing a cell-containing composition for delivery to a patient, the method comprising culturing the composition of claim 1 with biological cells, wherein the cells become associated with the plurality of threads to form the cell-containing composition.

22. A cell-containing composition made by the method of claim 21.

23. A method of treating a patient who has ischemic or necrotic tissue, the method comprising administering the composition of claim 2 to the ischemic or necrotic tissue by piercing the tissue and drawing the composition into the tissue.

24-25. (canceled)

26. A method of delivering a biological cell to cardiac tissue in need of repair, the method comprising:
(a) providing a biopolymer thread comprising one or more biological cells, wherein the thread is attached to a surgical needle and at least a portion of the thread is encased within a sleeve;
(b) drawing the needle through a region of the cardiac tissue to insert the sheath within the tissue; and
(c) removing the needle and sheath, thereby retaining the biopolymer thread in the tissue.

27. A method of making a tissue repair composition, the method comprising:
(a) providing cells that induce or enhance regeneration of tissue, wherein the cells are placed into a culture medium comprising a polymer thread having a leading end and a trailing end;
(b) culturing the cells under conditions that allow the cells to associate with the thread; and
(c) removing the thread and associated cells from the culture medium.

28. The method of claim 27, further comprising the step of encasing the polymer thread within a sleeve.

29. The method of claim 27, further comprising the step of attaching the leading end of the polymer thread to a needle.

30-34. (canceled)

35. A tissue repair composition made by the method of claim 29.
